(19) United States
(12) Patent Application Publication
(10) Pub. No.: US 2003/0125656 A1
(43) Pub. Date: Jul. 3, 2003

(54) Hemo-and biocompatible beaded polymeric material for purification of physiological fluids of organism, method of producing the material, as well as method of and device for purification of physiological fluids of organism with use of the material

(76) Inventors: Vadim Davankov, Moscow (RU); Maria Tsyurupa, Moscow (RU); Ludmila Pavlova, Moscow (RU); Ilya Zborovsky, Dix Hills, NY (US)

Correspondence Address: ILYA ZBOROVSKY, 6 Schoolhouse Way, Dix Hills, NY, 11746, US

(21) Appl. No.: 10/029,869

(22) Filed: Dec. 31, 2001

(51) Int. Cl. A61M 37/00; B01D 33/46

(52) U.S. Cl. 604/5.01; 210/263

(57) ABSTRACT

A hemo-and bio compatible beaded polymeric adsorbing material for purification of physiological fluids of organism has a plurality of beads each having a core with a hydrophobic core surface, and a hydrophilic, hemo-and biocompatible coating applied on the core surface of the core, so that the hemo-and biocompatible coating is applied non-continuously so as to leave on the core surface of the core such areas which are not covered with the hemo-and biocompatible coating and therefore remain hydrophobic, with the areas having a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid; and the material is produced by a new method, and also used for a method of and in a device for purification of physiological fluids of organism.

### BACKGROUND OF THE INVENTION

The present invention relates to biocompatible and hemocompatible polymeric adsorbents having a hydrophobic porous interior and a hydrophilic outer covering, as well as to methods of preparing the adsorbents and also to methods of and devices for purification of physiological fluids of organism with the use of the adsorbents.

Porous hydrophobic natural and polymeric materials, in particular, activated carbon and polymeric resins are widely used in adsorption technologies. They present a good choice for purifying blood or other physiological fluids of organism from many endogenic and exogenic toxic organic compounds. However, because of the high adsorption activity of the surface of the particles of these materials, the hydrophobic materials activate the blood complement system, cause deposition of platelets, and finally lead to clot formation. Therefore, in procedures for purification of physiological fluids of organism, only surface modified particles of the adsorbents can be employed. The modification is performed by forming a surface layer or coating of a hydrophilic biocompatible material, which however decreases the rate of diffusion of toxins into the interior of the adsorbing particle.

The materials which have a hydrophobic interior or core and hydrophilic biocompatible coating or shell are disclosed for example in U.S. Pat. Nos. 4,410,652; 4,202,775; 5,773,384; 5,904,663; 6,087,300; 6,114,466; 6,127,311; etc. The application of the coating on the surface of the core of the beads of the material is performed by various methods which involve formation of the hydrophilic biocompatible shell and its retention on the surface of the core. It is believed that the above-mentioned solutions can be further improved.

### SUMMARY OF THE INVENTION

Accordingly, it is an object of the present invention to provide a hemo-and biocompatible beaded polymeric material for purification of physiological fluids of organism, method of producing the material, as well as method of and device for purification of physiological fluids of organism with use of the material, which are further improvements of the existing solutions.

In keeping with these objects and with others which will become apparent hereinafter, one feature of present invention resides, briefly stated, in a hemo- and biocompatible beaded polymeric adsorbing material which comprises a plurality of beads each having a core with a hydrophobus core surface, and a hydrophilic, hemo-and biocompatible coating applied on the core surface of the core, the hemo-and biocompatible coating being applied non-continuously so as to leave on the core surface of the core such areas which are not covered with the hemo-and biocompatible coating and therefore remain hydrophobic, and the areas having a size which is substantially smaller than a size of an individual cell of the physiological fluid, so that when the physiological fluid passes through the material the individual cell of the physiological fluid can substantially be in contact only with the hemo- and biocompatible coating and can not contact the hydrophobic core surface of the core because the corresponding areas of the core surface which are exposed between parts of the hemo-and biocompatible hydrophilic coating have a smaller size than the individual cell of the physiological fluid.

In accordance with another feature of the present invention, a method of producing a hemo- and biocompatible beaded polymeric adsorbing material for purification of physiological fluids of organism, comprising the steps of forming cores of beads having a hydrophobic core surface; coating the core surface of the core of the beads with a hydrophillic hemo- and biocompatible coating, the hemo- and biocompatible coating being applied non-continuously so as to leave on the core surface of the core such areas which are not covered with the hemo- and biocompatible coating and therefore remain hydrophobic, and the areas having each a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

In accordance with the present invention a method of purification of physiological fluids of organism is proposed which includes passing a physiological fluid of organism through a hemo-and bio compatible beaded polymeric adsorbing material which has a plurality of beads each having a core with a hydrophobic core surface, and a hydrophilic, hemo-and biocompatible coating applied on the core surface of the core, the hemo- and biocompatible coating being applied non-continuously so as to leave on the core surface of the core such areas which are not covered with the hemo-and biocompatible coating and therefore remain hydrophobic, with the areas having each a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

Finally, a device for purification of physiological fluids of organism is proposed which has a container having inlet means, outlet means and an interior; and a body of a hemo-and bio compatible beaded polymeric adsorbing material which has plurality of beads each having a core with a hydrophobic core surface, and a hydrophilic, hemo-and biocompatible coating applied on the core surface of the core, the hemo- and biocompatible coating being applied non-continuously so as to leave on the core surface of the core such areas which are not covered with the hemo- and biocompatible coating and therefore remain hydrophobic, and of the areas having each a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid, so that when a physiological fluid passes from the inlet means to the outlet means through the interior of the container, only toxins from the physiological fluid are adbsorbed by the core surface of the core between portions of the hemo-and biocompatible coating and the purified physiological fluids leaves the container through the outlet means.

When purification of a physiological fluid of organism is performed in accordance with the inventive method, and/or in the inventive device, with the material formed and produced in accordance with the present invention, then the physiological fluids of organism which passes through the material is purified from toxins and at the same time the cells of blood are not negatively affected since they substantially do not contact the exposed hydrophobic core surface of the core of the beads, while toxic molecules during passage of the physiological fluids of organism are adsorbed by these areas.

The term “cell” is used here to define for a physiological liquid for example, for blood, natural, substantially healthy benign, cell of blood, such as erythrocytes, platelets, white blood cells, etc., as opposed to toxins which are unnatural, unhealthy, and damaging substances of a smaller molecular size.

The novel features which are considered as characteristic for the present invention are set forth in particular in the appended claims. The invention itself, however, both as to its construction and its method of operation, together with additional objects and advantages thereof, will be best understood from the following description of specific embodiments.

### DESCRIPTION OF THE PREFERRED EMBODIMENTS

In accordance with the present invention, a material is proposed for purification of physiological fluids, such as blood. The inventive material is a hemo-and biocompatible beaded polymeric material. It is composed of a plurality of beads each having a porous core with a hydrophobic core surface, and a hydrophilic, hemo-and biocompatible coating applied on the core surface of the core. The hemo-and biocompatible coating being applied non-continuously so as to leave on the core surface of the core such areas which are not covered with the hemo-and biocompatible coating and therefore remain hydrophobic. These areas have each a size which is substantially smaller than a size of an individual cell of the physiological fluid. Therefore so that when the physiological fluid passes through the material, the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

Each of the areas of the core surface of the core which are exposed and not covered by the hemo-and biocompatible hydrophillic coating can have the size which is 10-20% smaller than the size of the cells of the physiological fluid of organism.

The material can be also formed such that each of the areas of the core surface of the core which are exposed and not covered by the hemo-and biocompatible hydrophillic coating have the size which is smaller than the size of a smallest of the cells of the physiological fluid of organism.

The material in particular can be formed such that the size of each of the areas of the core surface of the core exposed between portions of hemo- and biocompatible coating for the physiological fluid which is blood is less than 1 micron.

In accordance with a further embodiment of the present invention, the material can be formed such that the areas of the core surface of the core which are exposed and not covered by the hemo- and biocompatible coating have each a size which is greater than a size of toxins in the physiological fluid of organism. The toxins thereby have the sufficient areas between the parts of the hemo- and biocompatible coating to reach and to be adsorbed by the hydrophilic core surface.

The material can be formed such that the areas of the core surface of the core which are exposed and not covered by the hemo- and biocompatible coating have each a size which is greater by 5-10% than a size of toxins in the physiological fluid of organism.

The material can be formed such that the areas of the core surface of the core exposed between portions of the hemo-and biocompatible hydrophillic coating for the physiological fluid which is blood have the size greater than 10 nm.

The hemo-and biocompatible beaded polymeric material in accordance with the present invention is produced by a method which includes the steps of forming cores of beads having hydrophobic core surface; coating the core surface of the beads with a hemo- and biocompatible hydrophillic coating; applying the hemo- and biocompatible coating non-continuously so as to leave on the core surface of said core such areas which are not covered with the hemo- and biocompatible coating and therefore remain hydrophobic, and selecting said areas with a size which is substantially smaller than a size of an individual cell of the physiological fluid. Therefore, as explained above when the physiological fluid passes through the material the individual cell of the physiological fluid can substantially be in contact only with the hemo-and biocompatible coating and can not contact the hydrophobous core surface of the core because the corresponding areas of the core surface which are exposed between parts of the hemo-and biocompatible hydrophilic coating each have a smaller size than the individual cell of the physiological fluid.

The application of the hemo-and biocompatible hydrophillic coating is performed so as to provide the areas of the hydrophobic core surface of the hydrophobic core between the portions of the hemo-and biocompatible hydrophillic coating with the sizes specified herein above.

For purification of a physiological fluid of organism with the use of the above mentioned material, a physiological fluid of organism is passed through a hemo-and bio compatible beaded polymeric adsorbing material which has a plurality of beads each having a core with a hydrophobic core surface, and a hydrophilic, hemo-and biocompatible coating applied on said core surface of said core, wherein the hemo- and biocompatible coating is applied non-continuously so as to leave on the core surface of said core such areas which are not covered with the hemo- and biocompatible coating and therefore remain hydrophobous, and have a size which is smaller than the size of the individual cell of the physiological liquid.

The purification of a physiological fluid of organism can be performed in a device which has a container with inlet means, outlet means and an interior; and a body of a hemo-and bio compatible beaded polymeric adsorbing material composed of beads each having a core with a hydrophobic core surface, and a hydrophilic, hemo-and biocompatible coating applied on said core surface of said core, with the hemo- and biocompatible coating being applied non-continuously so as to leave on the core surface of said core areas which are not covered with the hemo- and biocompatible coating and therefore remain hydrophobic, so that said areas have a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

As described above, in order to provide the hemo-and biocompatible hydrophillic coating on the core surface of the hydrophobic core, the size of the hydrophobic exposed areas should be less than the size of formular elements of blood, i.e. blood cells. Provided that this requirement is fulfilled, about half of the hydrophobic core surface may remain uncoated and easily accessible to toxic components. This allows the amount of the coating material to be reduced by a factor 1.5-2, simultaneously enhancing substantially the efficiency of blood purification.

In the material in accordance with the present invention which is used for example for purification of blood the size of each of the areas of the core surface of the hydrophobic core between the portions of the hemo-and biocompatible hydrophillic coating can be less than 1 micron. This size is smaller than the size of the smallest cells of blood, platelets that usually measure 1.5-2.5 micron.

The physiological liquids of organism can contain various toxins, depending on conditions or sicknesses of the organism. For example, blood can contain a series of middle molecular weight toxins in a patient with a renal disease, one of most examined being beta-2 microglobulin, a protein with molecular weight 11,800 Da and a diameter 33 angstrom.

As mentioned above, the size of the areas of the core surface of the hydrophobic core between the portions of the hemo- and biocompatible hydrophillic coating has to be smaller than the size of the smallest cell of blood, so that none of the blood cell can interact with and be adsorbed by the hydrophobic, exposed core surface areas. At the same time, the size of each of the exposed areas of the core surface of the hydrophobic core can be at least equal to or greater than the size of the largest toxin to be removed from the blood, so that the toxins of all sizes in the blood of this patient have sufficient exposed areas of the core surface of the hydrophobic core to interact with these areas and to be adsorbed for them. For example in the material for purification of blood, the size of each of the areas of the hydrophobic core surface can be 5-10% greater than the size of the toxins.

The examples for the inventive hemo- and biocompatible polymeric adsorbing material are presented herein below.

### EXAMPLE 1

Into a seven-liter four-necked round-bottom flask equipped with a stirrer, a thermometer and a reflux condenser, is placed the solution of 8.4 g polyvinyl alcohol-type technical grade emulsion stabilizer GM-14 in four liters of deionized water (aqueous phase). The solution of 260 ml divinylbenzene, 140 ml ethylvinylbenzene, with porogens 250 ml toluene and 250 ml n-octane, and 2.94 g benzoyl peroxide (organic phase) is then added to the aqueous phase on stirring at room temperature. In 20 min, the temperature is raised to 80° C. The reaction is carried out at 80° C. for 8 hours and 90-92° C. for additional 2 hours. After accomplishing the copolymerization, the stabilizer is rigorously washed out with hot water (60 to 80° C.). The liquid was removed from the reactor and the solution of 5 g Trisodium phosphate in 3 L water was added. When the temperature is raised to 80° the solution of 10.2 g of ammonium persulfate in hot water was added and in a few minutes the solution of 1.8 ml of N-vinyl-2-pyrrolidone in 100 ml H₂O was introduced. Reaction occurred 3 hours at 70° on stirring. After accomplishing the reaction polymer was washed with water and the above organic solvents are removed by steam distillation. The beads obtained are filtered, washed with 1 L dioxane and with deionized water. Finally, the beads are dried in oven at 60° C. overnight.

The Polymer Obtained in Example

1. displayed apparent inner surface area of 1200 sq.m/g and total pore volume of 0.8 ml/g,

2. increased its volume in ethanol by a factor of 1.3,

3. efficiently removed beta2-microglobuline from blood of patients on permanent dialysis treatment,

4. Individual spherical beads of the polymer of 0.4-0.63 mm in diameter were mechanically destroyed at a loading of 450±50 g, which is much better as compared to typical macroporous beads (about 120-150 g), but not as good as typical hypercrosslinked beads (up to 600 g) of a comparable diameter and total porous volume.

### EXAMPLE 2

As in Example 1, taking 220 ml divinylbenzene, 180 ml ethylvinylbenzene, porogens-150 ml toluene and 150 ml n-octane and 3.0 g benzoyl peroxide as the organic phase, 7.0 ml N-vinyl-2-pyrrolidone in aqueous phase. Inner surface area of the product obtained amounts to 1000 sq.m/g. Volume swelling with ethanol amounts to 1.25.

### EXAMPLE 3

As in Example 1, taking organic phase consisting of 320 ml divinylbenzene, 80 ml ethylvinylbenzene, porogens-600 ml toluene and 600 ml n-octane, and 2.94 g bis-azoisobuthyro nitrile, 3.0 ml N-vinyl-2-pyrrolidone in aqueous phase. Inner surface area of the product obtained amounts to 1150 sq.m/g. Volume swelling with ethanol amounts to 1.5.

### EXAMPLE 4

As in Example 3 conducting the polymerization at 800 for 6 hours. Then, the solution of 6 g trisodium phosphate in 40 ml of water, the solution of 10 g, ammonium persulfate in 20 ml H₂O and the solution of 2 ml N-vinyl-2-pyrrolidone in 20 ml water are added successively.

The reaction keeps going at 80° for 2 hours. The beads are washed with hot water, iso-propanol, water and dried. In accordance with analysis 1% of taken N-vinyl-2-pyrrolidone was grafted to the beads. The remaining fraction of N-vinyl-2-pyrrolidone was polymerized in solution.

### EXAMPLE 5

As in Example 1, taking 200 ml ethylene dichloride and 120 ml n-hexane as the porogen. Inner surface area of the product obtained amounts to 1000 sq.m/g. Volume swelling with ethanol amounts to 1.3.

### EXAMPLE 6

7.2 L of water was placed in 14 L glass vessel equipped with a stirrer and a reflux condenser and gradually heated to 80° C. When the temperature reached 60° C., 13.0 g of stabilizer, Airvol 523, were added. The stabilizer was dissolved within 40 min on stirring. Then 14.0 g of monosodium phosphate, 46.8 g of disodium phosphate, 28.7 g of trisodium phosphate, 72 g of sodium chloride and 150 mg of sodium nitrite were added. After complete dissolution of the chemicals the solution of 11.1 g of benzoyl peroxide in 935 ml of divinylbenzene, 765 ml of ethylstyrene, with porogen-1600 ml of iso-octane and 1120 ml of toluene was dispersed in the above aqueous phase. After 15 hours of stirring at 80° C. the aqueous phase is removed and replaced with a solution of 54,2 ml of N-vinyl-2-pyrrolidone and 25 g ammonium persulfate in 5000 ml of water. The surface modification of the polymer beads was afterwards carried out for 5 hours at 80° C. Upon accomplishing the reaction, beads were washed rigorously with hot water, methanol and cold water. The beads were filtered off and dried in oven at 60 to 80° C. Inner surface area of the polymer amounted to 650 m²/g, average pore size was 200 Å.

### EXAMPLE 7

7.2 L of water were placed in 14 L glass vessel equipped with a stirrer and a reflux condenser and heated to 80° C. When the temperature reached 60° C. 13.0 g of stabilizer, Airvol 523, were added. The stabilizer was dissolved within 40 min on stirring. Then 14.0 g of monosodium phosphate, 46.8 g of disodium phosphate, 28.7 g of trisodium phosphate, 72 g of sodium chloride and 150 mg of sodium nitrite were added. After complete dissolution of the chemicals the solution of 11.1 g of benzoyl peroxide in 1720 ml of 55% divinylbenzene, with porogen-1600 ml of iso-octane and 1120 ml of toluene was dispersed in the above aqueous phase. In 3 hours of stirring at 80° C. the solution of 15 ml of N-vinyl-2-pyrrolidone in 200 ml of water was added. The polymerization was carried out for 6 hours at 80° C. Upon accomplishing the reaction, beads were washed rigorously with hot water, methanol and cold water. The beads were filtered off and dried in oven at 60 to 80° C. Inner surface area of the polymer amounted to 650 m²/g, average pore size was 230 Å, the polymer was easily wetted with water. The presence of the grafted polyvinylpyrrolidone is further corroborated by the absorption amide band at about 1640 cm⁻¹ in the IR spectrum of the material.

### EXAMPLE 8

4.9 L of water were placed in a 14 L glass vessel equipped with a stirrer and a reflux condenser and heated to 80° C. When the temperature reached 60° C. 12.0 g of stabilizer, Airvol 523, were added. The stabilizer was dissolved within 40 min on stirring. Then 9.1 g of monosodium phosphate, 30.3 g of disodium phosphate, 17.3 g of trisodium phosphate, 47.0 g of sodium chloride and 100 mg of sodium nitrite were added. After complete dissolution of the chemicals the solution of 18.6 g of benzoyl peroxide in 945 ml of divinylbenzene, 655 ml of ethylstyrene, with porogen-1500 ml of iso-octane and 1000 ml of toluene was dispersed in the above aqueous phase. After 12 hours of stirring at 80° C., 27.3 g of ammonium persulfate were introduced into the aqueous phase. In 5 min the solution of 19.6 ml of N-vinyl-2-pyrrolidone in 100 ml of water was added. The polymerization was additionally carried out for 3 hours at 80° C. Upon accomplishing the reaction, beads were washed rigorously with hot water, methanol and cold water. The beads were filtered off and dried in oven at 60 to 80° C. Inner surface are of the polymer amounted to 650 m²/g, the polymer was wetted with water.

### EXAMPLE 9

5 L of water were placed in a 14 L glass vessel equipped with a stirrer and a reflux condenser and heated to 80° C. When the temperature reached 60° C. 12.0 g of stabilizer, Airvol 523, were added. The stabilizer was dissolved within 40 min on stirring. Then 9.1 g of monosodium phosphate, 30.3 g of disodium phosphate, 17.3 g of trisodium phosphate, and 100 mg of sodium nitrite were added. After complete dissolution of the chemicals the solution of 18.6 g of benzoyl peroxide in—1500 ml of 63% divinylbenzene, 1500 ml of iso-octane and 1000 ml of toluene was dispersed in the above aqueous phase. In 12 hours of stirring at 80° C. 27.3 g of ammonium persulfate were introduced into aqueous phase. In 10 min the solution of 41 g of acrylamide in 100 ml of water was added. The polymerization was additionally carried out for 3.5 hours at 80° C. Upon accomplishing the reaction, beads were washed rigorously with hot water, methanol and cold water. The beads were filtered off and dried in oven at 60 to 80° C. The polymer is wetted with water.

### EXAMPLE 10

5 L of water were placed in a 14 L glass vessel equipped with a stirrer and a reflux condenser and heated to 80° C. When the temperature reached 60° C. 12.0 g of stabilizer, Airvol 523, were added. The stabilizer was dissolved within 40 min on stirring. Then 25 g of sodium carbonate and 200 mg of sodium nitrite were added. After complete dissolution of the chemicals the solution of 18.6 g of benzoyl peroxide in 1500 ml of 63% divinylbenzene, with porogen-1500 ml of iso-octane and 1000 ml of toluene was dispersed in the above aqueous phase. In 12 hours of stirring at 80° C. 27.3 g of ammonium persulfate were introduced into the aqueous phase. In 5 min the solution of 41 g of 2-hydroxyethyl methacrylate in 150 ml of water were added. The polymerization was carried out for 3 hours at 80° C. Upon accomplishing the reaction, beads were washed rigorously with hot water, methanol and cold water. The beads were filtered out and dried in oven at 60 to 80° C. The polymer is wetted with water.

### EXAMPLE 11

7.2 L of water were placed in a 14 L glass vessel equipped with a stirrer and a reflux condenser and heated to 80° C. When the temperature reached 60° C. 13.0 g of stabilizer, Elvanol 523, were added. The stabilizer was dissolved within 40 min on stirring. Then 9.1 g of monosodium phosphate, 30.3 g of disodium phosphate, 17.3 g of trisodium phosphate, 47.0 g of sodium chloride and 100 mg of sodium nitrite were added. After complete dissolution of the chemicals the solution of 11.1 g of benzoyl peroxide in 1720 ml of 55% divinylbenzene, with porogen 1600 ml of iso-octane and 1120 ml of toluene was dispersed in the above aqueous phase. In 12 hours of stirring at 80° C. the temperature was lowered to 40° C. and the solution of 40.6 g ammonium persulfate in 100 ml of water was added. In several minutes 35 ml of tetramethyl ethylene diamine were introduced and afterwards the solution of 54,2 ml of N-vinyl-2-pyrrolidone in 200 ml of water was added. The grafting was carried out for 2 hours at 40° C. Upon accomplishing the reaction, beads were washed rigorously with hot water, methanol and cold water. The beads were filtered off and dried in oven at 60 to 80° C. The polymer is wetted with water.

### EXAMPLE 12

As in Example 11, but instead of 35 ml TEMED, 15.0 g trisodium phosphate were used.

### EXAMPLE 13

5 L of water were placed in a 14 L glass vessel equipped with a stirrer and a reflux condenser and heated to 60° C. At that temperature 12.0 g of stabilizer, Elvanol 523, were added. The stabilizer was dissolved within 40 min on stirring. Then 25 g of sodium carbonate and 200 mg of sodium nitrite were added. After complete dissolution of the chemicals the solution of 13.5 g of Vazo-52 in 800 ml of styrene, 700 ml of 63% divinylbenzene, with porogen 1500 ml of cyclohexane was dispersed in the above aqueous phase. In 4 hours of stirring at 60° C. the solution of 41 g of 2-hydroxyethyl methacrylate in 150 ml of water were added. The polymerization was carried out for 4 hours at 60° C. Upon accomplishing the reaction, beads were washed rigorously with hot water, methanol and cold water. The beads were filtered off and dried in oven at 60 to 80° C. Inner surface area of the polymer amounts to 880 m²/g, the polymer contains micropores of about 20 Å and mesopores of about 200 Å in diameter, the polymer is wetted with water.

### EXAMPLE 14

5 L of water were placed in a 14 L glass vessel equipped with a stirrer and a reflux condenser and heated to 80° C. When the temperature reached 60° C. 14.0 g of stabilizer, Elvanol 523, were added. The stabilizer was dissolved within 40 min on stirring. Then 35 g of sodium carbonate and 200 mg of sodium nitrite were added. After complete dissolution of the chemicals the solution of 20 g of benzoyl peroxide in 900 ml of n-buthyl methacrylate, 700 ml of 63% divinylbenzene, with porogen 1250 ml of toluene was dispersed in the above aqueous phase. In 2 hours of stirring at 80° C. the solution of 41 g of 2-hydroxyethyl methacrylate in 100 ml of water was added. The polymerization was carried out for 9 hours at 80° C. Upon accomplishing the reaction, beads were washed rigorously with hot water, methanol and cold water. The beads were filtered off and dried in oven at 60 to 80° C. The polymer is wetted with water.

### EXAMPLE 15

5 L of water were placed in a 14 L glass vessel equipped with a stirrer and a reflux condenser and heated to 80° C. When the temperature reached 60° C. 15.5 g of stabilizer, Airvol 523, were added. The stabilizer was dissolved within 40 min on stirring. Then 25 g of sodium carbonate and 200 mg of sodium nitrite were added. After complete dissolution of the chemicals the solution of 20 g of benzoyl peroxide in 945 ml of divinylbenzene, 555 ml of ethylstyrene, with porogen-3000 ml of iso-octane was dispersed in the above aqueous phase. In 4 hours of stirring at 80° C. the solution of 41 g of 2-hydroxyethyl methacrylate and 3 g of ammonium persulfate in 150 ml of water were added. The polymerization was carried out for 3 hours at 80° C. Upon accomplishing the reaction, beads were washed rigorously with hot water, methanol and cold water. The beads were filtered off and dried in oven at 60 to 80° C. Inner surface area of the polymer amounts to 560 m²/g, average pore size of macropores amounts to 350 Å, the polymer is wetted with water.

### EXAMPLE 16

7.2 L of water were placed in a 14 L glass vessel equipped with a stirrer and a reflux condenser and heated to 80° C. When the temperature reached 60° C. 13.0 g of stabilizer, Airvol 523, were added. The stabilizer was dissolved within 40 min on stirring. Then 46.8 g of disodium phosphate, 28.7 g of trisodium phosphate, and 150 mg of sodium nitrite were added. After complete dissolution of the chemicals the solution of 11.1 g of benzoyl peroxide in 1500 ml of trivinylbenzene, with porogen-1000 ml of iso-octane and 1000 ml of toluene was dispersed in the above aqueous phase. In tree hours of stirring at 80° C. the solution of 54,2 ml of N-vinyl-2-pyrrolidone and 2 ml of divinyl sulfone in 200 ml of water were added. The polymerization was afterwards carried out for 9 hours at 80° C. Upon accomplishing the reaction, beads were washed rigorously with hot water, methanol and cold water. The beads were filtered off and dried in oven at 60 to 80° C. Inner surface area of the polymer is 900 m²/g. The polymer is wetted with water.

### EXAMPLE 17

7.2 L of water were placed in a 14 L glass vessel equipped with a stirrer and a reflux condenser and heated to 80° C. When the temperature reached 60° C. 13.0 g of stabilizer, Elvanol 523, were added. The stabilizer was dissolved within 40 min on stirring. Then 14.0 g of monosodium phosphate, 46.8 g of disodium phosphate, 28.7 g of trisodium phosphate, 72 g of sodium chloride and 150 mg of sodium nitrite were added. After complete dissolution of the chemicals the solution of 11.1 g of benzoyl peroxide in 900 ml of α-methylstyrene, 300 ml of diisopropenylbenzene, with porgens 1700 ml of heptane and 930 ml of toluene was dispersed in the above aqueous phase. In three hours of stirring at 80° C. the solution of 7.0 ml of N-vinyl-2-pyrrolidone in 200 ml of water was added. The polymerization was afterwards carried out for 9 hours at 80° C. Upon accomplishing the reaction, beads were washed rigorously with hot water, methanol and cold water. The beads were filtered off and dried in oven at 60 to 80° C. The polymer is wetted with water.

### EXAMPLE 18

5 L of water were placed in a 14 L glass vessel equipped with a stirrer and a reflux condenser and heated to 80° C. When the temperature reached 60° C. 15.5 g of stabilizer, Airvol 523, were added. The stabilizer was dissolved within 40 min on stirring. Then 20 g of sodium carbonate and 300 mg of sodium nitrite were added. After complete dissolution of the chemicals the solution of 20 g of benzoyl peroxide in 1000 ml of tert-buthyl methacrylate, 350 ml of ethyleneglycol dimethacrylate, with porogen-1800 ml of toluene was dispersed in the above aqueous phase. In 4 hours of stirring at 80° C. the solution of 41 g of 2-hydroxyethyl methacrylate in 150 ml of water was added. The polymerization was carried out for 3 hours at 80° C. Upon accomplishing the reaction, beads were washed rigorously with hot water, methanol and cold water. The beads were filtered off and dried in oven at 60 to 80° C. The polymer obtained contains mostly micropores of 10 to 20 Å in diameter and a small portion of mesopores around 150 Å. The polymer is wetted with water.

### EXAMPLE 19

50 ml of water is placed in a 100 ml vessel equipped with a stirrer and a reflux condenser and heated to 80° C. When the temperature is reached 0.2 g of Airvol 523 is added. After complete dissolution of the stabilizer 2 mg of sodium nitrite and 0.65 g of acrylamide are added. Afterwards the solution of 0.39 g of benzoyl peroxide and 13 g of pure p-divinylbenzene in porogen-16 ml of toluene is dispersed in the above aqueous phase. The polymerization is carried out for 9 hours at 80° C. Upon accomplishing the reaction, the beads obtained are washed with hot water, methanol and cold water and dried in oven at 60 to 80° C. The beads are wetted with water.

It will be understood that each of the elements described above, or two or more together, may also find a useful application in other types of methods and constructions differing from the types described above.

While the invention has been illustrated and described as embodied in hemo-and biocompatible beaded polymeric material for purification of physiological fluids of organism, method of producing the material, as well as method of and device for purification of physiological fluids of organism with use of the material, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A hemo-and bio compatible beaded polymeric adsorbing material for purification of physiological fluids of organism, comprising a plurality of beads each having a core with a hydrophobic core surface, and a hydrophilic, hemo-and biocompatible coating applied on the core surface of the core, the hemo- and biocompatible coating being applied non-continuously so as to leave on the core surface of the core such areas which are not covered with the hemo- and biocompatible coating and therefore remain hydrophobous, the areas having a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

2. A hemo-and bio compatible beaded polymeric adsorbing material as defined in claim 1, wherein each of said areas of the core surface of the core which are exposed and not covered by the hemo-and biocompatible hydrophillic coating have the size which is 10-20% smaller than the size of the cells of the physiological fluid of organism.

3. A hemo-and bio compatible beaded adsorbing material as defined in claim 1, wherein each of the areas of the core surface of the core which are exposed and not covered by the hemo-and biocompatible hydrophillic coating have the size which is smaller than the size of a smallest of the cells of the physiological fluid of organism.

4. A hemo-and bio compatible beaded polymeric adsorbing material as defined in claim 1, wherein the size of the areas of the core surface of the core exposed between portions of hemo- and biocompatible coating for the physiological fluid which is blood is less than 1 micron.

5. A hemo- and bio compatible beaded adsorbing material as defined in claim 1, wherein the areas of the core surface of the core which are exposed and not covered by the hemo- and biocompatible coating have each a size which is greater than a size of toxins in the physiological fluid of organism.

6. A hemo- and bio compatible beaded adsorbing material as defined in claim 5, wherein the areas of the core surface of the core which are exposed and not covered by the hemo- and biocompatible coating have each a size which is greater by 5-10% than the size of toxins in the physiological fluid of organism.

7. A hemo- and bio compatible beaded adsorbing material as defined in claim 7, wherein the areas of the core surface of the core exposed between portions of the hemo-and biocompatible hydrophillic coating for the physiological fluid which is blood each have the size greater than 10 nm.

8. A method of producing a hemo- and biocompatible polymeric adsorbing material for purification of physiological fluids of organism, comprising the steps of forming cores of beads having hydrophobic core surface; coating the core surface of the beads with a hemo- and biocompatible hydrophillic coating, so that the hemo- and biocompatible coating is applied non-continuously so as to leave on the core surface of the core areas which are not covered with the hemo- and biocompatible coating and therefore remain hydrophobic, and forming the areas with a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

9. A method as defined in claim 8, wherein said forming includes forming each of said areas of the core surface of the core which are exposed and not covered by the hemo-and biocompatible hydrophillic coating each have the size which is 10-20% smaller than the size of the cells of the physiological fluid of organism.

10. A method as defined in claim 8, wherein said forming includes forming each of the areas of the core surface of the core which are exposed and not covered by said hemo-and biocompatible hydrophillic coating each have the size which is smaller than the size of a smallest of the cells of the physiological fluid of organism.

11. A method as defined in claim 10, wherein said forming includes forming the size of each of the areas of the core surface of the core exposed between portions of the hemo- and biocompatible coating for the physiological fluid which is blood is less than 1 micron.

12. A method as defined in claim 11, wherein said forming includes forming the areas of the core surface of the core which are exposed and not covered by the hemo- and biocompatible coating have each a size which is greater than a size of toxins in the physiological fluid of organism.

13. A method as defined in claim 12, wherein said forming includes forming the areas of the core surface of the core which are exposed and not covered by the hemo- and biocompatible coating have each a size which is greater by 5-10% than a size of toxins in the physiological fluid of organism.

14. A method as defined in claim 17, wherein said forming includes forming the areas of the core surface of the core exposed between portions of the hemo-and biocompatible hydrophillic coating for the physiological fluid which is blood each have the size greater than 10 nm.

15. A method of purification of physiological fluids of organism, comprising the steps of passing a physiological fluid of organism through a hemo-and bio compatible beaded polymeric adsorbing material which has a plurality of beads each having a core with a hydrophobic core surface, and a hydrophilic, hemo-and biocompatible coating applied on the core surface of the core, with the hemo- and biocompatible coating being applied non-continuously so as to leave on the core surface of the core such areas which are not covered with the hemo- and biocompatible coating and therefore remain hydrophobic, and the areas each have a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.

16. A device for purification of physiological fluids of organism, comprising a container having inlet means, outlet means and an interior; and a body of a hemo-and bio compatible beaded polymeric adsorbing material which has a plurality of beads each having a core with a hydrophobic core surface, and a hydrophilic, hemo-and biocompatible coating applied on the core surface of the core, with the hemo- and biocompatible coating being applied non-continuously so as to leave on the core surface of the core such areas which are not covered with the hemo- and biocompatible coating and therefore remain hydrophobic, and the areas each having a size which is substantially smaller than a size of an individual cell of the physiological fluid, so that when the physiological fluid passes through the material the individual cell of the physiological fluid can substantially be in contact only with the hemo-and biocompatible coating and can not contact the hydrophobic core surface of the core because the corresponding areas of the core surface which are exposed between parts of the hemo-and biocompatible hydrophilic coating have a smaller size than the individual cell of the physiological fluid.
